(19) United States
(12) Patent Application Publication
(10) Pub. No.: US 2020/0060786 A1
(43) Pub. Date: Feb. 27, 2020

(54) OPERATING ROOM LAYOUT AND METHOD OF UTILIZING SAME

(71) Applicant: Optical Integrity, Inc., Panama City Beach, FL (US)

(72) Inventors: JOE D. BROWN, PANAMA CITY BEACH, FL (US); DANIEL MALPHURS, PANAMA CITY BEACH, FL (US)

(73) Assignee: OPTICAL INTEGRITY, INC.

(21) Appl. No.: 16/546,810

(22) Filed: Aug. 21, 2019

Related U.S. Application Data
(60) Provisional application No. 62/720,341, filed on Aug. 21, 2018.

(51) Int. Cl. A61B 90/00; A61B 18/20; A61G 13/00; G06F 3/14

(52) U.S. Cl. CPC A61B 90/37; A61B 18/20; A61B 2017/00221; G06F 3/1423; A61G 13/00

(57) ABSTRACT

The need for persons to move around a crowded operating room during a surgical procedure is reduced by providing each of the persons tasked with operating surgical equipment, such as a laser, with a portable or handheld communication device capable of transmitting instructions and commands necessary to operate the laser, and by further providing each person in the operating room whose duties require observing a monitor with display devices capable of displaying video and other information shown on the monitor. The handheld wireless communication devices may be smartphones or tablets, and the display devices may be separate wireless display devices or displays of the smartphones or tablets. Operation of the surgical equipment may be by voice commands, allowing the equipment to be controlled while continuously viewing the video and information on the display device

This application claims the benefit of U.S. Provisional Patent Appl. Ser. No. 62/720,341, filed Aug. 21, 2018, and incorporated herein by reference.

### BACKGROUND OF THE INVENTION

### 1. Field of the Invention

The invention relates to an improved operating room layout, and in particular to an operating room for carrying out surgical laser procedures such as cystoscopy, in which a doctor, nurse, and/or surgical assistant can control the laser and monitor the surgical procedure while minimizing the need to move around the operating room.

The invention also relates to a method of utilizing the improved operating room layout to operate a laser and monitor a surgical laser procedure from anywhere in the operating room.

### 2. Description of Related Art

Operating rooms are crowded places. A cystoscopy operating room, for example, includes laser equipment, scope monitoring equipment, anesthesia equipment, and personnel including a doctor, surgical assistant, anesthesiologist, and a nurse. An example of such an operating room, without the personnel, is shown in FIG. 1. The clutter and lack of open space in the operating room can make it difficult to safely carry out certain tasks during a surgical procedure.

For example, the job of the nurse is to monitor progress of the procedure, turn on and adjust power to the laser under instructions from the doctor, and provide the doctor or surgical assistant with equipment whenever it is needed. Because the monitor is typically located on one side of the operating room, the laser on another side, and the surgical supply area and nurse's station on yet another side, the nurse is required to move about the operating room. In a crowded operating room, this can be difficult to accomplish without imperiling the patient by bumping into equipment or other personnel, tripping over wires or laser fibers, or crossing over the surgical field.

It has previously been proposed to facilitate laser operation by utilizing a smartphone or tablet to control the laser. As disclosed in U.S. Patent Publication No. 2018/0042676, the smartphone or tablet is used to input control instructions to the laser, and to display laser operating parameters. However, no provision is made for monitoring patient data utilizing the handheld device. In addition, attempting to input instructions on the relatively small touchscreen of a smartphone or tablet can lead to input errors, which may be compounded by the operator's need to divide his attention between the screen of the handheld device, which displays laser parameters, and the monitor that displays patient information.

### SUMMARY OF THE INVENTION

It is an objective of the invention to provide an operating room layout and method that allows greater efficiency and safety during a surgical laser procedure such as cystoscopy.

It is also an objective of the invention to provide an improved operating room layout and method that allows the doctor, surgical assistant and nurse to carry out duties involving operation of the laser and monitoring a surgical procedure without having to move around the operating room.

These objectives are achieved by providing each of the persons in an operating room tasked with operating surgical equipment, such as a laser, with a portable or handheld communication device capable of transmitting instructions and commands necessary to operate the laser, and further providing each person in the operating room whose duties require observing a monitor with display devices capable of displaying video and other information shown on the monitor. The handheld wireless communication devices may be smartphones or tablets, and the display devices may either be separate wireless display devices or displays of the smartphones or tablets.

In an especially advantageously embodiment of the invention, operation of the surgical equipment may be by voice commands, allowing the equipment to be controlled while continuously viewing the video and information on the display device.

### BRIEF DESCRIPTION OF THE DRAWINGS

FIG. 1 is perspective view of a portion of a conventional operating room, illustrating the crowded nature of the operating room.

FIG. 2 is schematic view of an operating room similar to that of FIG. 1, set up for a laser surgery procedure.

FIG. 3 is schematic view showing an improvement to the operating room of FIGS. 1 and 2, in which personnel are provided with wireless devices for operating a laser and monitoring a surgical procedure from anywhere in the operating room in accordance with the principles of a preferred embodiment of the invention.

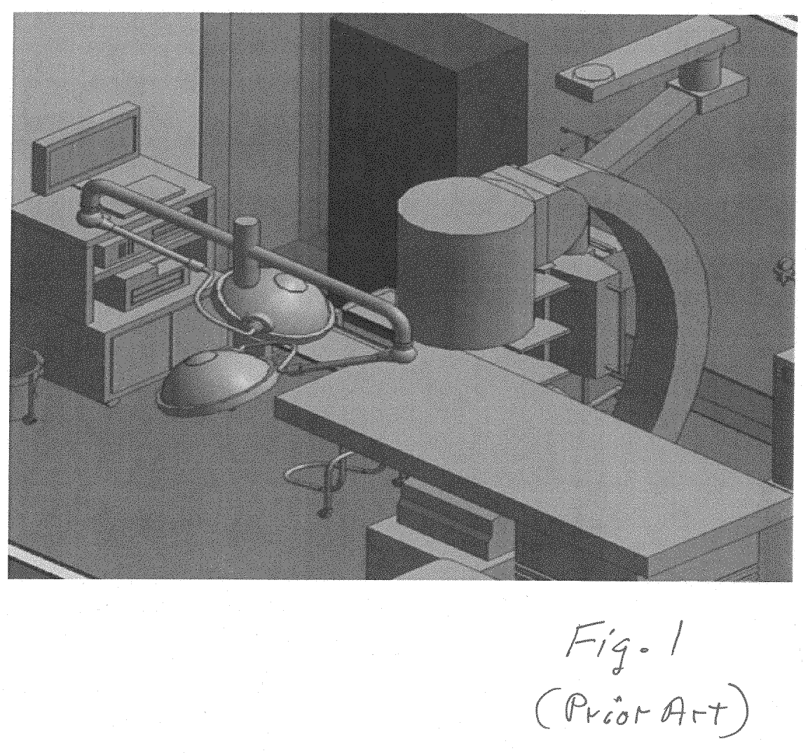

### DETAILED DESCRIPTION OF THE PREFERRED EMBODIMENTS

Throughout the following description and drawings, like reference numbers/characters refer to like elements. It should be understood that, although specific exemplary embodiments are discussed herein there is no intent to limit the scope of present invention to such embodiments. To the contrary, it should be understood that the exemplary embodiments discussed herein are for illustrative purposes, and that modified and alternative embodiments may be implemented without departing from the scope of the present invention.

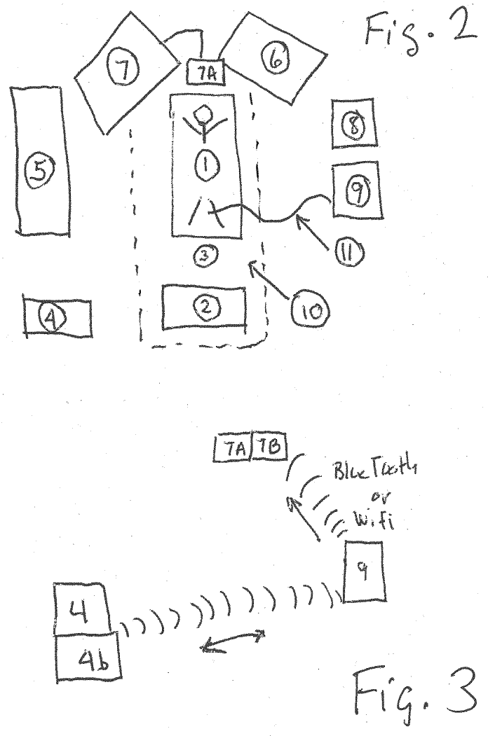

As illustrated in FIG. 2, an operating room in which surgical laser procedures such as cystoscopy are performed includes an operating table 1, a sterile table 2 on which are placed sterile items during the surgical procedure, a nurse station 4, a surgical supply storage table or cabinet 5, an anesthesia station 6, and an x-ray machine 8. In addition, in order to perform cystoscopy or similar procedures, the operating room includes a laser 9, a laser fiber 10 extending from the laser 9, and a video station 7 provided with a monitors 7. Reference numeral 10 indicates a sterile area surrounding the operating table 1. When the surgeon is at a position 3 near the foot of the operating table, he or she is in a position to carry out a procedure using the laser fiber 10.

In order to maintain sterility, it is necessary for the doctor and/or surgical assistant to stay at the position 3 located at the foot of the operating table while the procedure is being performed. Furthermore, according to the principles of the invention, a nurse is able to stay at the nurse station 4, which is within reach of the position 3, in order to be in a position to assist and hand surgical supplies to the doctor or surgical assistant whenever needed. There is no need for the surgeon or nurse to traverse the crowded operating room to approach the video station 7 and possibly interfere with the anesthesiologist at anesthesiology station 6.

The reason that there is no need for the doctor, surgical assistant, and nurse to move away from their respective positions 3 and 4 is that, as illustrated in FIG. 3, each of the persons in the operating room that is responsible for controlling equipment such as the laser 9 or observing the monitor 7 is provided with a handheld communication device 7b such as a tablet or smartphone, and a wireless display device 4b, which may be a display of the handheld communication device. The tablet and smartphone may, for example, be an iPad™ or iPhone™. The tablet or smartphone are capable of wirelessly communicating instructions or commands for controlling the laser 9. The display device 4b receives wireless communications from the video station 7, in order to display information or video shown on the monitor 7 and/or other information useful during a surgical procedure.

The wireless communications may be carried out by Bluetooth®, WiFi, WiFi Voice, or any other wireless communications protocol suitable for use in a relatively confined space, and having sufficient bandwidth to enable display device 4b to mirror the video and information shown on the monitor 7.

If voice communications are provided for, the laser or other equipment is preferably provided with a voice activation code or name to avoid unintentional commands. For example, the nurse may activate the laser by beginning with the word “Joe,” followed by appropriate commands such as “on” and power level. This has the advantage of allowing continuous observation of the monitor even while controlling the laser.

What is claimed is:

1. An operating room layout, the operating room including an operating table, a video monitor, and surgical equipment, comprising
at least one at least one communication device provided to each person in the operating room tasked with operating surgical equipment capable of transmitting instructions and commands necessary to operate the laser; and
and at least one display device provided to each person in the operating room whose duties require observing the monitor,
wherein the at least one handheld communication device wirelessly transmits instructions and commands to the surgical equipment, and
wherein the at least one handheld display device is adapted to display video and other information shown on the monitor,
thereby reducing a need for the persons to move about the operating room.

2. An operating room layout as claimed in claim 1, wherein the surgical equipment is a surgical laser.

3. An operating room layout as claimed in claim 1, wherein the at least one handheld communication device is a tablet or smartphone.

4. An operating room layout as claimed in claim 3, wherein the at least one display device is a separate display device.

5. An operating room layout as claimed in claim 3, wherein the at least one display device is a display of the tablet or smartphone.

6. An operating room layout as claimed in claim 1, wherein wireless communications between the at least one handheld communication device, surgical equipment, at least one handheld display device, and a video station for the monitor are carried out by Bluetooth®, WiFi, or WiFi Voice.

7. An operating room layout as claimed in claim 1, wherein the surgical equipment is operated by voice commands and instructions received from the at least one handheld communication device.

8. A method of reducing a need to move around an operating room during a surgical procedure, comprising steps of:
operating surgical equipment by commands and instructions wirelessly transmitted from a handheld communication device; and
transmitting video and information displayed on a monitor from a video station and to at least one handheld display device for display on the display device.

9. A method as claimed in claim 8, wherein the surgical equipment is a surgical laser.

10. A method as claimed in claim 8, wherein the at least one handheld communication device is a tablet or smartphone.

11. A method as claimed in claim 10, wherein the at least one display device is a separate display device.

12. A method as claimed in claim 10, wherein the at least one display device is a display of the tablet or smartphone.

13. A method as claimed in claim 8, wherein wireless communications between the at least one handheld communication device, surgical equipment, at least one handheld display device, and a video station for the monitor are carried out by Bluetooth®, WiFi, or WiFi Voice.

14. A method as claimed in claim 8, wherein the step of operating the surgical equipment comprises the step of issuing voice commands and instructions to the portable surgical equipment.
